(19) United States
(12) Patent Application Publication
(10) Pub. No.: US 2021/0371821 A1
(43) Pub. Date: Dec. 2, 2021

(54) INDUCED REGULATORY T CELLS, METHODS OF PRODUCTION, AND USES THEREOF

(71) Applicant: THE HENRY M. JACKSON FOUNDATION FOR THE ADVANCEMENT OF MILITARY MEDICINE, INC., Bethesda, MD (US)

(72) Inventor: Yong Chan Kim, Rockville, MD (US)

(21) Appl. No.: 17/335,808

(22) Filed: Jun. 1, 2021

Related U.S. Application Data
(60) Provisional application No. 63/033,080, filed on Jun. 1, 2020.

(51) Int. Cl. C12N 5/0783; A61K 35/17

(52) U.S. Cl. CPC C12N 5/0637; A61K 35/17; C12N 2501/2302; C12N 2501/15; C12N 2500/40

(57) ABSTRACT

The present disclosure provides methods for producing cell populations enriched for induced regulatory T cells (iTregs). In particular, the disclosure relates to methods for culturing T cells such that the final culture is enriched for induced regulatory T cells. The disclosure also relates to methods for inducing regulatory T cells. Also provided are compositions enriched for induced regulatory T cells, which are useful for treating individuals in need of such treatment. The methods and compositions disclosed herein can also be used to treat individuals suffering from immune-mediated diseases.

### CROSS-REFERENCE TO RELATED APPLICATIONS

This application claims benefit of the priority date of U.S. Appl. No. 63/033,080, filed Jun. 1, 2020, which is incorporated by reference herein in its entirety.

### FIELD OF INVENTION

The present invention relates to induced regulatory T cells, methods of producing the induced regulatory T cells, and uses of the induced regulatory T cells.

### BACKGROUND

Foxp3⁺ Tregs are a unique subset of CD4⁺ T cells responsible for self-tolerance and for the prevention of autoimmune disease (Shevach E M, Immunity, 2009; 30(5):636-645). Adoptive Treg infusion has been suggested as a potential therapy for the prevention of Graft versus Host Disease (GVHD) following stem cell transplantation, organ allograft rejection, and for the treatment of autoimmune diseases such as type I diabetes and multiple sclerosis (Roncarolo M-G, Battaglia M., Nat Rev Immuno., 2007; 7(8):585-598; Riley J L, June C H, Blazar B R, Immunity, 2009; 30(5):656-665). Adoptive transfer of Foxp3⁺ Tregs in mouse models has been shown to prevent acute and chronic GVHD without negative effects on the graft versus leukemia response (Hoffmann P, Ermann J, Edinger M, Fathman C G, Strober S, J Exp Med, 2002; 196(3):389-399). More recently, a number of groups have reported that co-transfer of expanded Tregs from umbilical cord samples (Brunstein C G, Miller J S, Cao Q, et al., Blood, 2011; 117(3):1061-1070) or from peripheral blood appears to be both safe (Trzonkowski P, Bieniaszewska M, Jukinska J, et al., Clin Immunol, 2009; 133(1):22-26) and in one study remarkably effective in preventing acute GVHD following stem cell transplantation (Di Ianni M, Falzetti F, Carotti A, et al., Blood, 2011; 117(14):3921-3928).

Although considerable enthusiasm has been generated for adoptive Treg therapy, several major issues remain to be resolved. First, most clinical applications of Treg therapy will require large numbers of cells and optimal methods for Treg expansion are now being explored. Expansion of highly purified populations of human Tregs also frequently results in loss of Foxp3 expression during the expansion process. Secondly, in contrast to studies in the mouse, Foxp3 expression can be readily induced during in vitro stimulation of conventional human T cells (Shevach E M, Tran D Q, Davidson T S, Andersson J, Eur J Immunol, 2008; 38(4):915-917). However, T cells induced in vitro to express Foxp3 frequently lack a Treg phenotype, continue to make effector cytokines and lack in vitro suppressive function (Shevach E M, Tran D Q, Davidson T S, Andersson J, Eur J Immunol, 2008; 38(4):915-917). Thus, expression of Foxp3 cannot be considered a completely reliable marker for functional human Tregs.

A number of approaches have been used to address these problems. Combined use of several surface markers (CD127^{lo} and CD25^{hi}) has facilitated isolation of more highly enriched populations of Foxp3⁺ T cells with less contamination by CD25^{int} activated T cells (Liu W, Putnam A L, Xu-Yu Z, et al., J Exp Med. 2006; 203(7):1701-1711). Addition of inhibitors of the mTOR pathway, such as rapamycin, block the expansion of contaminating conventional T cells and favor the expansion of Tregs, but purity greater than 60% is rarely achieved after several rounds of expansion depending on the starting population (Hippen K L, Merkel S C, Schirm D K, et al., American Journal of Transplantation, 2011; 11(6): 1148-1157). CD4⁺CD25⁺CD45RA⁺Foxp3⁺ T cells, although a minor subpopulation (5-30%) of the Foxp3⁺ pool in adults, appear to have a greater propensity to expand in culture and have enhanced stability of Foxp3 expression compared to CD4⁺CD25⁺CD45RO⁺Foxp3⁺ T cells (Miyara M, Yoshioka Y, Kitoh A, et al., Immunity, 2009; 30(6):899-911).

Foxp3⁺ Tregs can be divided into two potentially distinct subpopulations. One population is generated in the thymus and has been termed natural (n)Tregs. A second population is generated extrathymically in peripheral sites and has been termed induced (i) Tregs or adaptive Treg. It has recently (Thornton A M, Korty P E, Tran D Q, et al., J. Immunol. 2010; 184(7):3433-3441) been demonstrated that the transcription factor, Helios, a member of the Ikaros gene superfamily, is expressed in 70% of both mouse and human Foxp3⁺ T cells.

### SUMMARY OF INVENTION

Disclosed herein are methods of producing an immunosuppressive iTreg (induced regulatory T cell), comprising treating an isolated CD3⁺CD4⁺CD25^{-/lo}CD127⁺Foxp3⁻Helios⁻ T cell with an isolated oligodeoxynucleotide (ODN) having a phosphorothioate backbone and TGFβ1. The methods can further comprise treating the T cell with IL-2.

In some embodiments, the ODN is 11-49 nucleotides in length. In some embodiments, the ODN is 21-25 nucleotides in length. In other embodiments, the ODN is 25 nucleotides in length.

The methods produce iTreg that is CD3⁺CD4⁺CD25^{-/+}Foxp3⁺Helios^{-/lo} and immunosuppressive. In some embodiments, the iTreg expression of IFNγ is lower compared to treatment with TGFβ1 alone or ODN alone.

Also disclosed herein are iTregs produced by the methods disclosed herein. Also disclosed are iTregs (induced regulatory T cell) that are CD3⁺CD4⁺CD25^{-/+}Foxp3⁺Helios^{-/lo} and immunosuppressive. In some embodiments, the iTreg expression of IFNγ is lower compared to treatment with TGFβ1 alone or ODN alone.

Also disclosed herein are compositions comprising the iTregs disclosed herein and a carrier. Also disclosed herein are compositions comprising an isolated CD3⁺CD4⁺CD25^{-/lo}CD127⁺Foxp3⁻Helios⁻ T cell, an isolated ODN having a phosphorothioate backbone, and TGFβ1. The compositions can further comprise IL-2.

Disclosed herein are kits comprising an isolated CD3⁺CD4⁺CD25^{-/lo}CD127⁺Foxp3⁻Helios⁻ T cell and an isolated ODN having a phosphorothioate backbone. The kits can further comprise TGFβ1. The kits can further comprise IL-2.

Disclosed herein are methods of treating an autoimmune disease in a subject in need thereof, comprising administering to the subject the iTregs disclosed herein. The autoimmune disease can be but is not limited to Type I diabetes, multiple sclerosis, Graft vs. host disease, allograft rejection, atopic dermatitis, psoriasis, inflammatory bowel disease, neuromyelitis optica, rheumatoid arthritis, alopecia areata, systemic lupus erythematosus, pemphigus vulgaris, autoimmune vasculitis, xenogeneic organ transplantation, allogenic organ transplantation, or ADA (anti-drug antibody)-mediated complications.

### BRIEF DESCRIPTION OF FIGURES

The present disclosure can be better understood by reference to the following drawings. The drawings are merely exemplary to illustrate certain features that may be used singularly or in combination with other features and the present disclosure should not be limited to the embodiments shown.

FIGS. 1A-1B. Flow cytometry sorting strategy. Data was derived from human CD4⁺ T cells. which were enriched from PBMCs by CD4⁺ selection using Human CD4 MicroBeads. The bead-isolated cells were sorted on a FACSAria™ flow cytometer using FACSDiva™ software. Before sort (FIG. 1A): Doublet discrimination were performed by using FSC-A and FSC-H parameters followed by “Lymphocyte Gate” for expected morphology using F SC-A vs. SSC-A. Next, CD4⁺ lymphocytes were gated followed by gating the naive T cells (CD4⁺CD25^{lo/-}, CD127⁺CD45RA⁺) and regulatory T (Treg, CD4⁺CD25^{hi}CD127^{lo/-}) cells. CD4⁺CD25^{hi}CD127^{lo/-}, here called nTregs, were used as positive control for further experiment. After Sorting (FIG. 1B): Sorted cells were further analyzed for their purity with the same gating strategy.

FIG. 2. A scheme of the experimental setup. FACS-sorted CD4⁺ T cells were pre-stimulated with anti-CD³/anti-CD28 antibodies and IL-2 for 2 days and transduced with 17195 virus by spinfection on the retronectin-coated virus plate. Next day, infected cells were activated with gamma-irradiated HLA-DR1 PBMCs, cognate FVIII C2 peptide-2191-2220 (pC2), and IL-2 until day 5 in the conditions as indicated. After day 5, the cells were resuspended in fresh media and IL-2 and the cells were continuously cultured up to day 10. On day 10, the cells were activated again with the same way with antigen specific TCR stimuli and IL-2 in the condition as indicated until day 15.

FIGS. 3A-3D. The effect of TGFβ1 and ODN on iTreg phenotype. FACS-sorted naïve T cells were pre-stimulated with anti-CD³/anti-CD28 antibodies and IL-2 in the absence (PBS) or presence of TGFβ1 and/or ODN, as indicated. The cells were transduced with retrovirus containing the TCR-17195-IRES-GFP, which is the retrovirus containing the TCR-17195 linked with green fluorescent protein (GFP) via intra ribosome entry site (IRES), and then activated with γ-irradiated PBMCs, pC2, and IL-2 along with the absence (PBS) or presence of TGFβ1 and/or ODN, as indicated. On day 5, the cells were analyzed for intracellular Foxp3 and Helios. FACS-sorted nTregs were transduced with the same retrovirus described above and used as a positive control. FIG. 3A. Lymphocyte gating for expected morphology using forward versus side scatter. FIG. 3B. Gating strategy for transduced GFP⁺CD4⁺ T cells expressing TCR-17195 (17195 T cells). FIG. 3C. Intracellular staining for Foxp3 and Helios in 17195 T cells. FIG. 3D. Histogram plots for intracellular Foxp3 and Helios in 17195 T cells.

FIGS. 4A-4D. Phenotype analysis of Treg markers and cytokine secretion analysis by flow cytometry after 2^{nd} expansion. T cells expended in the first round were re-stimulated on day 10 with 1 μg/ml of pC2 and irradiated PBMCs in the indicated condition in the presence of IL-2 for up to 15 days, and then flow cytometry analyses were performed. FIG. 4A. Gating strategy for GFP⁺ cells. FIG. 4B. Staining of Foxp3 and Helios in GFP⁺17195 T cells. FIG. 4C. Second-round expanded 17195TCR-transduced Tregs were rested for 3 days of culture without IL-2, and then re-stimulated for 4 hours with phorbol myristate acetate (PMA) and ionomycin in the presence of Golgi-block reagent. Intracellular IFNγ and IL-2 levels were measured by FACs analysis. FIG. 4D. Histogram analyses of GFP⁺17195 T cells.

FIGS. 5A-5B. Flow cytometry sorting strategy for naïve T cell and Treg isolation. Human CD4⁺ T cells were enriched from PBMCs using Human CD4 MicroBeads. The bead-isolated cells were sorted on a BD FACSMelody™ Cell Sorter, as the indicated gating strategy. Before sorting (FIG. 5A): Lymphocytes were gated by expected morphology using forward versus side scatter. Next, CD3⁺CD4⁺ lymphocytes were gated and followed by gating the naïve T cells (CD3⁺CD4⁺CD25^{-/lo}CD127⁺CD45RA⁺) and regulatory T (Treg, CD3⁺CD4⁺CD25^{hi}CD127^{-/lo}) cells. CD3⁺CD4⁺CD25^{hi}CD127^{-/lo}, here called natural Tregs (nTregs), were used as a positive control for further experiment. After Sorting (FIG. 5B): Sorted cells were further analyzed for their purities with the indicated gating strategy.

FIG. 6. Experimental outline of iTreg generation. FACS-sorted naïve T cells were pre-stimulated with anti-CD³/anti-CD28 antibodies, IL-2, ODN, and TGFβ1 for 2 days and then transduced with retrovirus containing the TCR called as 17195, which is the FVIII-2191-2220-specific TCR, by spinfection on the retronectin-coated plate. On day 3, infected cells were activated with γ-irradiated HLA-DR1 PBMCs, cognate FVIII C2 peptide-2191-2220 (pC2), and IL-2 until day 5. On day 5, the cells were resuspended in fresh media containing IL-2 and continuously cultured until day 10. On day 10, the cells were restimulated again with the same method used on day 3 until day 15. The phenotype of cells was analyzed on day 5 and 15 using a flow cytometry.

FIGS. 7A-7D. Phenotype analysis of iTreg by flow cytometry after 2^{nd} expansion. Pre-stimulated naïve T cells were transduced with retrovirus containing the TCR-17195-IRES-GFP on day 2 and then activated with γ-irradiated HLA-DR1 PBMCs, pC2, and IL-2 in the absence (PBS) or presence of TGFβ1 and/or ODN, as indicated, on day 3 and 10. The cells were expanded for 15 days, and then flow cytometry analyses were performed. FIG. 7A. Gating strategy for transduced GFP⁺CD4⁺17195 T cells. FIG. 7B. Staining of intracellular Foxp3 and Helios in 17195 T cells. FIG. 7C. Histogram analyses for intracellular Foxp3 and Helios of 17195 T cells. FIG. 7D. Phenotype analysis of iTreg (prepared as in FIG. 6).

FIGS. 8A-8C. Characterization of immunosuppressive functions in iTreg. FIG. 8A. 17195TCR-transduced T cells were expanded for 15 days as described in FIG. 6. in the absence (PBS) or presence of TGFβ1 and/or ODN, as indicated. The cells were rested for 3 days without IL-2, and then re-stimulated for 4 hours with phorbol myristate acetate (PMA) and ionomycin in the presence of Golgi-block reagent. Intracellular IFN-γ and IL-2 expressions were measured by FACs analysis. FIG. 8B. 17195TCR-transduced iTregs were generated from 4 different donors in the absence (PBS) or presence of TGFβ1 either alone (TGFβ1) or together with ODN (TGFβ1+ODN). IFNγ-expressing GFP⁺Foxp3⁺ cells were measured by FACs analysis. nTregs transduced with 17195TCR were used as a positive control. Results were analyzed using a one-tailed t test (*P<0.05, ***P<0.0001). FIG. 8C. 17195TCR-transduced iTregs were generated in the absence (PBS) or presence of TGFβ1 alone (TGFβ1) or together with ODN (TGFβ1+ODN). 17195TCR-transduced T effectors (Teff) were co-cultured with 17195TCR-transduced iTregs or nTregs (Treg) at different Teff/Treg ratios in the presence of the FVIII peptide (pC2 2191-2220) and γ-irradiated PBMCs for 4 days without IL-2. Immunosuppression by Tregs was evaluated using [³H]-thymidine incorporation assay. Data are a representative of three experiments with different 3 donors.

### DETAILED DESCRIPTION OF THE INVENTION

Disclosed herein are methods of producing immunosuppressive iTregs (induced T regulatory cells) that are CD3⁺CD4⁺CD25^{-/+}Foxp3⁺Helios^{-/lo} and useful for treating diseases related to the immune system. Such populations of cells are useful for preventing or treating diseases such as Graft versus host Disease (GVHD) and autoimmune diseases such as, for example, type I diabetes, multiple sclerosis, and allograft rejection following tissue transplantation.

Before the present disclosure is further described, it is to be understood that this disclosure is not strictly limited to particular embodiments described herein, as such can of course vary. It is also to be understood that the terminology used herein is for the purpose of describing particular embodiments only, and is not intended to be limiting, since the scope of the present disclosure will be limited only by the claims.

It must be noted that as used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the detailed methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates, which may need to be independently confirmed.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments are specifically embraced by the present disclosure and are disclosed herein just as if each and every combination was individually and explicitly disclosed. In addition, all sub-combinations are also specifically embraced by the present disclosure and are disclosed herein just as if each and every such sub-combination was individually and explicitly disclosed herein.

It is further noted that the claims can be drafted to exclude any optional element. As such, this statement is intended to serve as antecedent basis for use of such exclusive terminology as “solely,” “only” and the like in connection with the recitation of claim elements or use of a “negative” limitation.

Accordingly, disclosed herein are methods of producing an immunosuppressive iTreg (induced T regulatory cell), e.g., an isolated human immunosuppressive iTreg, comprising treating an isolated CD3⁺CD4⁺CD25^{-/lo}CD127⁺Foxp3⁻Helios⁻ T cell with an isolated oligodeoxynucleotide (ODN) having a phosphorothioate backbone and TGFβ1 to generate the immunosuppressive iTreg. In some embodiments, the methods further comprise treating the CD3⁺CD4⁺CD25^{-/lo} CD127⁺Foxp3⁻Helios⁻ T cell with IL-2.

Also disclosed are immunosuppressive iTregs that are CD3⁺CD4⁺CD25^{-/+}Foxp3⁺Helios^{-/lo}. Also disclosed are compositions comprising the iTreg disclosed herein and a carrier. In some embodiments, the iTregs can be isolated and/or human iTregs. The iTregs are not Helios⁺, e.g., the iTregs are not CD3⁺CD4⁺CD25^{-/lo} Foxp3⁺Helios⁺, CD3⁺CD4⁺CD25⁺Foxp3⁺Helios⁺, or CD3⁺CD4⁺CD25^{-/lo}Foxp3⁺Helios⁺.

In some embodiments, the immunosuppressive iTreg expression of IFNγ is lower when the CD3⁺CD4⁺CD25^{-/lo}CD127⁺Foxp3⁻Helios⁻ T cell is treated with TGFβ1 and ODN compared to treatment of the CD3⁺CD4⁺CD25^{-/I}TD127⁺Foxp3⁻Helios⁻ T cell with TGFβ1 alone or ODN alone.

As used herein, the term “immunosuppressive” refers to reducing the activation or efficacy of the immune system.

As used herein, the terms “isolated,” “isolating,” “purified,” and the like, do not necessarily refer to the degree of purity of a cell or molecule of the present disclosure. Such terms instead refer to cells or molecules that have been separated from their natural milieu or from components of the environment in which they are produced. For example, a naturally occurring cell or molecule (e.g., a T cell, a DNA molecule, etc.) present in a living animal, including humans, is not isolated. However, the same cell, or molecule, separated from some or all of the coexisting materials in the animal, is considered isolated. As a further example, according to the present disclosure, cells that are present in a sample of blood obtained from a person would be considered isolated. It should be appreciated that cells obtained from such a sample using further purification steps would also be referred to as isolated, in keeping with the notion that isolated does not refer to the degree of purity of the cells.

With further regard to the disclosure herein, isolated cells useful for practicing the disclosed methods can be any isolated cells that comprise T cells. Such cells can be obtained as a sample from an animal, including humans, or they can be obtained from cells in culture. Examples of cell samples useful for practicing the present disclosure include, but are not limited to, blood samples, lymph samples, and tissue samples. In one embodiment, the isolated cells are obtained from a blood sample. In another embodiment, the isolated cells are obtained from cells in culture.

It is known in the art that T cells belong to the class of cells known as lymphocytes, which are a type of agranulocyte. Agranulocytes, also known as mononuclear leukocytes, are characterized by the absence of granules in their cytoplasm. The lymphocytes comprise at least three separate cell types: B-cells, T cells and natural killer cells. As used herein, “T cells” include effector T cells (also generally termed “T cells”), regulatory T cells (also termed “T regulatory cells” or “Tregs”), and induced regulatory T cells (iTregs). In various embodiments, the isolated cells can comprise mononuclear, agranulocyte or lymphocyte cell populations, so long as they comprise T cells, and in particular, T cells that are CD3⁺CD4⁺CD25^{-/lo}CD127⁺Foxp3⁻Helios⁻, as a starting material.

As used herein, Tregs are a subpopulation of T cells that suppress activation of the immune system and express, at least, the transcription factor Foxp3. Tregs suppress cytokine production and proliferation of T effector cells. Tregs do not express pro-inflammatory cytokines such as interferon-gamma, interleukin-17, and interleukin-2, and do not proliferate when stimulated via the T cell receptor in vitro in the absence of IL-2. Tregs are generally Helios⁺, e.g., CD3⁺CD4⁺CD25^{-/lo}Foxp3⁺Helios⁺, CD3⁺CD4⁺CD25⁺Foxp3⁺Helios⁺, or CD3⁺CD4⁺CD25^{hi} Foxp3⁺Helios⁺. However, the iTregs disclosed herein are Helios⁺ or Helios^{lo}, e.g., CD3⁺CD4⁺CD25^{-/+}Foxp3⁺Helios⁻, CD3⁺CD4⁺CD25^{-/+}Foxp3⁺Helios^{lo}, or CD3⁺CD4⁺CD25^{-/+}Foxp3⁺Helios^{-/lo}.

As used herein, “induced” regulatory T cells or iTregs mean T regulatory cells expressing CD3⁺CD4⁺CD25^{-/+}Foxp3⁺Helios^{-/lo} by treatment of CD3⁺CD4⁺CD25^{-/lo} CD127⁺Foxp3⁻Helios⁻ T cells with TGFβ1 and ODN, or TGFβ1, ODN, and IL-2.

Several methods are used to identify, isolate, or obtain T cells, Tregs, and/or iTregs. Such methods include Fluorescent Activated Cell Sorting (FACS), e.g., FACSAria™, BIC, USUHS, based on the cell surface markers, such as but not limited to CD3, CD4, CD25, Foxp3, and Helios. In some embodiments, T cells, Tregs, and/or iTregs can be isolated or obtained by BD FACSMelody™ or FACSAria™ II cell sorter. In some embodiments, the iTregs include those with little or no expression of Helios (Helios^{-/lo}) and CD4⁺CD25^{+/hi}Foxp3⁺. In accordance with any of these embodiments, the iTregs can maintain its phenotype after transduction and long-term expansion.

For example, all Tregs express the CD4 and CD25 proteins, and thus are CD4⁺ and CD25⁺. Such proteins are therefore referred to as markers, or marker proteins, for Tregs. Thus, in some embodiments, the isolated cells comprise Tregs that are at least CD4⁺CD25⁺. Such cells make up about 5-10% of the mature CD4⁺ T cell population in humans, and about 1-2% of CD4⁺ cells in whole blood. However, because the CD25 protein can also be expressed on non-regulatory cells during activation of the immune system, a more accurate identification of Tregs in a cell population can be made by detecting expression of the transcription factor protein, forkhead box p3 (Foxp3). Thus, in some embodiments, the isolated cells comprise Tregs that are at least CD4⁺CD25⁺Foxp3⁺. A small percentage of Tregs can express Foxp3, but express low to undetectable levels of CD25. Detection of the presence or absence of other marker proteins can improve this analysis even further. Such markers include, for example, Helios (a member of the Ikaros family of zinc finger proteins) and CD127. With regard to CD127, the absence or low (lo) levels of expression of this protein, as compared to intermediate (int) or high (hi) levels of expression, indicates the T cell is a Treg. Methods of determining whether the expression level of CD127 is low, intermediate, or high, are disclosed herein and are known to those skilled in the art. For example, Liu W, Putnam A L, Xu-Yu Z, et al. CD127 expression inversely correlates with FoxP3 and suppressive function of human CD4⁺ T reg cells. J Exp Med. 2006; 203(7):1701-1711, which is incorporate herein by reference, teaches that low level-expression of CD127 is one of phenotypic feature of peripheral blood-resident Tregs in healthy donors and patients and allows one to distinguish Foxp3⁺ Treg from Foxp3⁻ effector cells. Accordingly, this reference teaches assays for measuring the level of expression of CD127.

As used herein, a marker that is “+” means the presence of the marker on the cell surface or inside the cell. A marker that is “−” means the absence of the marker on the cell surface or inside the cell. A marker that is “lo” means fewer expression of a marker relative to thymic natural Treg cells.

As used herein, “naïve T cells” refers to T cells that have differentiated in bone marrow and have not encountered its cognate antigen within the periphery. As used herein “natural Tregs” or “nTregs” mean bona fide Tregs produced by a normal thymus.

In various embodiments, the iTregs that have at least one characteristic selected from the group consisting of: (i) being Helios⁻, (ii) being CD127⁻; and, (iii) being CD127^{lo}. In some embodiments, the iTregs that are CD3⁺CD4⁺CD25^{+/-}Foxp3⁺Helios⁻. In further embodiments, the iTregs are CD3⁺CD4⁺CD25^{+/-}Foxp3⁺Helios⁻CD127⁻. In other embodiments, the iTregs are CD4⁺CD25^{+/-}Foxp3⁺Helios^{-/lo}.

As used herein, the term “stable” with regards to T cells refers to T cells that maintain expression of particular markers over multiple generations. For example, stable iTregs maintain expression of the specific iTreg markers disclosed herein, over several generations. In some embodiments, stable iTregs are those that maintain expression of CD4 and at least one marker selected from the group consisting of CD25⁺, Foxp3⁺, Helios⁻, Helios^{lo}, CD127⁻, and CD127^{lo}. In some embodiments, stable iTregs are those that remain CD4⁺CD25⁺Foxp3⁺ over multiple generations, e.g., but not limited to 2-5, 6-10, 11-20, or 21-30 generations or any number or ranges thereof. In some embodiments, stable iTregs are those that remain CD4⁺CD25⁺Foxp3⁺Helios^{-/lo} over multiple generations, e.g., but not limited to 2-5, 6-10, 11-20, or 21-30 generations or any number or ranges thereof. In some embodiments, stable iTregs are those that remain CD4⁺CD25⁺Foxp3⁺Helios^{-/lo} and CD127⁻ or CD127^{lo} over multiple generations, e.g., but not limited to 2-5, 6-10, 11-20, or 21-30 generations or any number or ranges thereof.

As used herein, the term “generation” refers to a round of replication. Thus, a cell that has divided one time has gone through one generation. If the progeny cells then divided once more, the original cells are considered to have gone through two generations of replication. The use of such terms is known by those it the art. In one embodiment, stable T cells are those maintain expression of markers of the present disclosure for at least about 10 generations. In one embodiment, stable T cells are those maintain expression of markers of the present disclosure for at least about 15 generations. In one embodiment, stable T cells are those maintain expression of markers of the present disclosure for at least about 20 generations. In one embodiment, stable T cells are those maintain expression of markers of the present disclosure for at least about 25 generations. In one embodiment, stable T cells are those maintain expression of markers of the present disclosure for at least about 30 generations. With regards to the number of generations, the term about is used for convenience and means plus or minus two generations.

The stable expression of markers can also be measured in days. Thus, in various embodiments, stable T cells are Tregs that maintain expression of markers of the present disclosure for at least about 10 days, at least about 15 days, at least about 20 days, at least about 25 days, or at least about 30 days. With regard to such measurement, the term “about” is used for convenience and means plus or minus two days.

According to the present disclosure, once isolated cells are obtained, they are cultured in the presence of an ODN having particular characteristics. It should be noted that isolated cells can be used directly in the culture step, or they can be further purified or concentrated prior to being cultured with an ODN. For example, T cells present in an isolated sample of cells can be identified using molecules, such as antibodies, that bind to T cell markers, thereby allowing the identification of T cells. The identified T cells can then be separated and pooled, or otherwise concentrated, to increase the concentration of T cells in the sample. Methods of concentrating cells are known to those skilled in the art and include, for example, flow cytometry and the use of columns containing molecules that bind Treg markers. In some embodiments, the concentration of T cells is increased by incubating the isolated cells with a molecule that binds T cells and then separating T cells from non-T cells by flow cytometry. In such embodiments, the molecules that bind T cell markers can be labeled with a detectable marker such as, for example, a florescent dye or a radiolabel. Suitable detectable markers are known to those skilled in the art.

As used herein, the term “oligodeoxynucleotide (ODN)” refers to a polymer of nucleotides (or bases). Such ODNs can be synthesized (e.g., using a nucleic acid synthesizer such as, for example, an Applied Biosystems Model 380B DNA synthesizer), or it can be generated by degradation (e.g., chemical or enzymatic digestion, shearing, etc.) of a larger nucleic acid molecule. While ODNs of the present disclosure can be any size capable of stimulating enrichment of Tregs in a population of isolated cells, ODNs having certain length characteristics, offer advantages over ODNs that are either longer or shorter. For example, ODNs that are too long result in a decrease in the viability of cells exposed to such ODNs. Moreover, ODNs that are too short do not stimulate enrichment of Tregs in a population of isolated cells. Thus, in some embodiments, the isolated ODNs of the present disclosure are less than about 300 nucleotides in length, less than about 200 nucleotides in length, less than about 100 nucleotides in length, or less than about 50 nucleotides in length. It should be noted that with regards to ODNs of the present disclosure, the term “about” means plus or minus 10%. Further, the isolated ODNs of the present disclosure should be at least 10 nucleotides in length. Thus, in some embodiments, the ODN is between 11 and about 199 nucleotides in length, the ODN is between about 15 and about 99 nucleotides in length, the ODN is between about 15 and about 50 nucleotides in length, the ODN is between about 20 and about 30 nucleotides in length, or mixtures thereof. In some embodiments, the ODN is an isolated ODN of 21 nucleotides in length, an isolated ODN of 22 nucleotides in length, an isolated ODN of 23 nucleotides in length, an isolated ODN of 24 nucleotides in length, an isolated ODN of 25 nucleotides in length, an isolated ODN of 26 nucleotides in length, an isolated ODN of 27 nucleotides in length, an isolated ODN of 28 nucleotides in length, an isolated ODN of 29 nucleotides in length, or mixtures thereof. In some embodiments, the ODN is 25 nucleotides in length.

ODNs of the present disclosure can have any sequence of nucleotides. That is, the ability of an ODN to stimulate enrichment of Tregs in a population of isolated cells is independent of its sequence. Thus, ODNs of the present disclosure can or cannot have a pattern. In some embodiments, the ODN consists of a mixture of ODNs having different sequences. As used herein, a mixture of ODNs having different sequence means that the order of the nucleotides was not chosen, by a person or machine (e.g., computer) to have a specific pattern, such as, for example, a protein encoding sequence, a binding site or a repeating sequence of nucleotides. That is, at each position in the ODN, there is an equal probability that any of the four possible nucleotides (i.e., adenine, guanine, cytosine and thymine) will be present. As noted above, while the ODN can have a mixture of ODNs having different sequences, it is not precluded from containing a pattern such as, for example, a repeating run of nucleotides, a protein encoding sequence, an endonuclease recognition site or a binding site. For example, inclusion of a binding motif within, or on the end of, an ODN can be useful in purification. Thus, in some embodiments, the ODN comprises a repeating pattern. In some embodiments, the ODN comprises a site selected from the group consisting of a biding motif and a restriction endonuclease recognition site. Similarly, the ODN is not precluded from being a polymer of a single type of nucleotide.

It should also be appreciated that ODNs of the present disclosure can be modified to improve, or confer, certain characteristics on the ODN. For example, modified ODNs can be more stable or have fluorescent properties. Such modifications can be made during synthesis of the ODNs or afterwards. For example, modified nucleoside triphosphates, such as alpha-phosphorothioates, 2′-O-methyl nucleotides, 7-Deazapurine nucleosides, or 2-aminopurine can be incorporated into the ODNs during synthesis. Methods of modifying nucleic acid molecules are disclosed in Verma and Eckstein, Modified ODNs Synthesis and Strategy for Users., Annu Rev. Biochem 1998. 67:99-134, which is hereby incorporated by reference. Thus, in some embodiments, the ODN is modified.

Use of the ODNs for stabilizing Tregs are described in U.S. Pat. No. 9,481,866, which is incorporated by reference herein in its entirety. ODN can be added at a concentration of but not limited to 0.1 μM to 10 μM, 0.2 μM to 10 μM, 0.3 μM to 10 μM, 0.4 μM to 10 μM, 0.5 μM to 10 μM to 1 μM to 9 μM, 1 μM to 8 μM, 1 μM to 7 μM, 1 μM to 6 μM, 1 μM to 5 μM, 1 μM to 4 μM, 1 μM to 3 μM, 1 μM to 2 μM, or any concentration or ranges therein.

Once a suitable ODN or a mixture of ODNs having different sequences has been obtained, it is cultured with isolated T cells of the present disclosure. According to the present disclosure, culturing (or incubating) the isolated cells in the presence of the ODN simply means that the ODN and the cells are brought together such that they are able to come into contact. Simply as an example of one method of achieving the goals of the disclosure, the cells could be placed into a vessel such as an EPPENDORF™ tube, along with the ODN. The mixture could be allowed to sit for a period of time to allow the ODN and the cells to come into contact, after which the mixture could be plated or introduced to culture bottles for growth. As an alternative example, the isolated cells and the ODN could be introduced directly into culture plates or bottles. Any such technique can be used, so long as the ODN and the isolated cells are allowed to come into contact.

Once the ODNs and the cells have been mixed, they are then cultured (or incubated) to allow expansion of at least a portion of the T cell population present in the isolated cells. Incubation can result in expansion of at least a portion of the T cell population present in the isolated cells. As used herein, expansion of a cell population means that at least one cell within a population is able to grow and divide, resulting in a population of cells retaining the characteristics of the original (progenitor) cell(s). Thus, for example, if a culture containing a single cell is expanded for five generations, the expanded culture will contain 32 (2⁵) cells. If the expanded cells are a stable population of cells, all 32 cells will retain the characteristics (e.g., express the same marker proteins, such as, CD4, CD25, Helios, Foxp3, etc) as the progenitor cell. General methods of culturing cells so that they grow and expand are known to those skilled in the art. Accordingly, it will be appreciated that culture conditions can vary depending on the types of cells being expanded, and/or the characteristics desired of the expanded cells. With regards to the present disclosure, the cells can be expanded in the presence of certain molecules that favor, or are necessary for, the expansion of T cells, and in particular iTregs. The requirement of the present method for inclusion of an ODN has already been described. In some embodiments, the isolated cells are expanded in the presence of at least one molecule selected form the group consisting of anti-CDR antibody, anti-CD28-antibody, interleukin-2 (IL-2), inhibitors of the mTOR pathway, rapamycin, functional analogs of the afore-mentioned molecules, and mixtures thereof.

In the presence of ODN and TGFβ1, the original T cells are induced to generate iTregs of the present disclosure. Moreover, the iTregs disclosed herein are immunosuppressive. TGFβ1 is a polypeptide member of the transforming growth factor beta superfamily of cytokines. TGFβ1 is a secreted protein that performs many cellular functions, including the control of cell growth, cell proliferation, cell differentiation, and apoptosis. TGFβ1 can be added at a concentration of but not limited to 0.1 ng/ml or great, 1 ng/ml or greater, 10 ng/ml or greater, 30 ng/ml or greater, 35 ng/ml or greater, 40 ng/ml or greater, 45 ng/ml or greater, or 50 ng/ml or greater, e.g., 0.1 ng/ml to 500 ng/ml, 1 ng/ml to 100 ng/ml, 10 ng/ml to 100 ng/ml, 30 ng/ml to 100 ng/ml, 35 ng/ml to 75 ng/ml, 40 ng/ml to 60 ng/ml, 35 ng/ml to 50 ng/ml, or any concentrations or ranges of concentrations therein. In some embodiments, the TGFβ1 concentration is not 25 ng/ml to 35 ng/ml.

In some embodiments, the T cells are further treated with IL-2 (interleukin-2). IL-2 is an interleukin, a type of cytokine signaling molecule in the immune system, which regulates the activities of leukocytes, often lymphocytes, that are responsible for immunity. IL-2 can be added in a concentration of but not limited to 0.1 IU/ml or greater, 1 IU/ml or greater, 10 IU/ml or greater, or 50 IU/ml or greater, e.g., 0.1 IU/ml to 5000 IU/ml, 1 IU/ml to 1000 IU/ml, 10 IU/ml to 500 IU/ml, 50 IU/ml to 100 IU/ml, or any concentrations or ranges of concentrations therein. In some embodiments, the IL-2 concentration is not 0.01 IU/ml to 0.07 IU/ml.

Once the appropriate incubation conditions have been established, the T cells are cultured so that the population of iTreg cells are induced, yielding a final population of cells that is enriched for induced regulatory T cells (iTregs). As has been previously discussed, only a small percentage of mature CD4⁺ T cells in humans are Tregs (natural Tregs). Moreover, while approximately 98% of Tregs present in blood retain their immunosuppressive function upon isolation from blood, following expansion of such cells using currently available methods, only about 15-20% of the expanded cells retain such function. However, the methods disclosed in U.S. Pat. No. 9,481,866 provide enriched populations of cells in which at least 50% or more of the cells are Tregs. As used herein, the term enriched, with respect to T cell populations, refers to a population of cells in which at least about 50% of the cells in the expanded cell population are stable Tregs. That is, at least 50% of the T cells in the population maintain the ability to suppress immune function. Thus, in some embodiments, at least about 50% of the T cells in the population are stable Tregs. In some embodiments, at least about 60% of the T cells in the population are regulatory T cells. In some embodiments, at least about 70% of the T cells in the population are stable Tregs. In some embodiments, at least about 75% of the T cells in the population are stable Tregs. In some embodiments, at least about 80% of the T cells in the population are stable Tregs. In some embodiments, at least about 85% of the T cells in the population are stable Tregs. In some embodiments, at least about 90% of the T cells in the population are stable Tregs. In some embodiments, at least about 95% of the T cells in the population are stable Tregs. Such methods can be used to maintain a stable population of iTregs.

As has been described, upon culture of Tregs isolated from blood, a large percentage of such cells lose markers associated with Tregs. Furthermore, described herein is how expansion of such cells in the presence of an ODN results in a culture enriched for stable Tregs. It will be appreciated by those skilled in the art that loss of Tregs during expansion of isolated cells could result from loss of expression of Treg markers, or failure of Treg cells, which by definition express such markers, to expand. Without being bound by theory, the ODNs may exert a direct effect on iTregs, thereby stabilizing, or maintaining, expression of Treg markers during the expansion of such cells. Accordingly, disclosed herein are methods to stabilize expression of iTreg markers and incubating the isolated cells in the presence of an ODN of the present disclosure, under conditions that result in the expansion of at least some of the initial, regulatory T cells. Such a method yields progeny iTreg cells that stably express iTreg markers. In some embodiments, the expanded iTregs stably express CD4 and at least one marker selected from the group consisting of CD3, CD25, Foxp3, and CD127^{lo}. In some embodiments, the expanded iTregs stably express CD3, CD4, CD25, and Foxp3 but do express Helios (Helios⁻ or Helios^{lo}).

Methods of the present disclosure result in the production of compositions having iTregs, which can be used for treating various disease related to the immune system. Prior to the discovery disclosed herein, such compositions were impractical, or even impossible, to produce, due to various factors such as cost and technical. Thus, disclosed herein are compositions comprising isolated T cells, wherein at least 50%, at least 60%, at least 70%, at least 80%, at least 90%, at least 95%, at least 99% of the T cells are iTregs. In some embodiments, the iTregs are positive for CD4 and at least one marker selected from the group consisting of CD3, CD25, Foxp3, CD127⁻, and/or CD127^{lo}. In some embodiments, iTregs are CD3⁺CD4⁺CD25⁺Foxp3⁺Helios^{-/lo}.

Because iTregs are able to suppress activation of the immune system, such cells can be used to treat an individual having a disease for which suppression of the immune system is desirable. Compositions of the present disclosure are particularly useful for treating autoimmune diseases. For example, Tregs can be used to treat or prevent a disease or condition such diabetes, multiple sclerosis, graft vs. host disease (GVHD) (e.g., after a bone marrow transplantation), allograft rejection following tissue transplantation, and the like. Thus, disclosed herein are methods to treat an individual in need of such treatment, the method comprising administering a composition comprising iTreg cells, wherein at least about 60% of the T cells are stable iTregs. In some embodiments, at least about 70% of the T cells in the composition are stable iTregs. In some embodiments, at least about 80% of the T cells in the composition are stable iTregs. In some embodiments, at least about 90% of the T cells in the composition are stable iTregs. In some embodiments, at least about 95% of the T cells in the composition are stable iTregs. In some embodiments, at least about 97% of the T cells in the composition are stable iTregs. In some embodiments, at least about 99% of the T cells in the composition are stable iTregs.

The compositions disclosed herein can further comprise carriers or excipients. In some aspects, the present disclosure provides pharmaceutical compositions suitable for pharmaceutical use comprising the iTregs and a pharmaceutically acceptable excipient or carrier. The compositions disclosed here can further comprise additional pharmaceutically active agents.

As used herein, “pharmaceutically acceptable” or “pharmacologically acceptable” mean molecular entities and compositions that do not produce an adverse, allergic or other untoward reaction when administered to an animal, or to a human, as appropriate.

The term, “pharmaceutically acceptable excipient or carrier” includes one or more inert excipients, which include water, buffer, starches, polyols, granulating agents, microcrystalline cellulose, diluents, lubricants, binders, disintegrating agents, and the like, and any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. “Pharmaceutically acceptable excipient” also encompasses controlled release means.

The composition, shape, and type of dosage form can typically vary according to applications thereof. For example, a dosage form suitable for mucosal administration can include a smaller amount of the active ingredient than that in a dosage form suitable for oral administration used in treating the same disease. These aspects of the present disclosure will be fairly apparent to those of ordinary skill in the art (reference: Remington's Pharmaceutical Sciences (1990) 18th ed., Mack Publishing, Easton Pa.).

Typical pharmaceutical compositions and dosage forms include one or more excipients. Suitable excipients are apparent to those of ordinary skill in the pharmaceutical art, and the present disclosure is not limited to examples of suitable excipients described herein.

Whether a particular excipient is suitable for a pharmaceutical composition or a dosage form depends on various factors well known in the art, including methods of formulating preparations to be administered to a patient, but is not limited thereto. For example, dosage forms for oral administration such as tablets can include an excipient not suitable for use in preparations for non-oral administration.

The pharmaceutical compositions include those suitable for aerosol, pulmonary, inhalation, oral, parenteral (including subcutaneous, intradermal, intramuscular, intravenous and intraarticular), rectal and topical (including dermal, buccal, sublingual and intraocular) administration. The most suitable route can depend upon the condition and disorder of the recipient. The compositions can conveniently be presented in unit dosage form and can be prepared by any of the methods well known in the art of pharmacy. In general, the compositions are prepared by uniformly and intimately bringing into association the active ingredient with liquid carriers or finely divided solid carriers or both and then, if necessary, shaping the product into the desired composition.

Compositions of the present disclosure can also optionally include other therapeutic ingredients, anti-caking agents, preservatives, sweetening agents, colorants, flavors, desiccants, plasticizers, dyes, and the like. Any such optional ingredient must, of course, be compatible with the compound of the disclosure to insure the stability of the composition.

The terms “individual,” “subject,” and “patient” are well-recognized in the art, and are herein used interchangeably to refer to a mammal, including dog, cat, rat, mouse, monkey, cow, horse, goat, sheep, pig, camel, and a human. In some embodiments, the subject has been diagnosed with an autoimmune disease. In some embodiments, compositions of the present disclosure are administered to an individual at risk for developing an autoimmune disease. Such risk can be due to, for example, genetic factors or exposure to environmental factors. Methods of identifying individuals at risk for developing an autoimmune disease are known to those in the art.

The terms individual, subject, and patient by themselves do not denote a particular age, sex, race, and the like. Thus, individuals of any age, whether male or female, are intended to be covered by the present disclosure.

Compositions of the present disclosure are administered using any known route used to administer therapeutic compositions, so long as such administration results in alleviation of symptoms of an autoimmune disease. Acceptable protocols by which to administer compositions of the present disclosure in an effective manner can vary according to individual dose size, number of doses, frequency of dose administration, and mode of administration. Determination of such protocols can be accomplished by those skilled in the art.

Also included in the present disclosure are kits useful for practicing the disclosed methods of the present disclosure. Thus, disclosed herein are kits comprising T cells (CD3⁺CD4⁺CD25^{-/lo}CD127⁺Foxp3⁻Helios) and isolated ODN having a phosphorothioate backbone. The kits can further comprise TGFβ1. The kits can further comprise IL-2. The kits can further comprise instructions for using the kit. Kits of the present disclosure can also comprise various reagents, such as buffers, necessary to practice the methods of the disclosure, as known in the art. Such reagents and buffers can, for example, be useful for establishing conditions appropriate for expanding isolated cells into enriched populations of Tregs. Thus, such regents can include things such as, for example, tissue culture media, immunoregulatory molecules such as TGFβ1 and/or IL-2.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The following examples are provided for the purpose of illustration and are not intended to limit the scope of the present disclosure.

### EXAMPLES

### Example 1. Materials and Methods

### General

Recombinant human interleukin (IL)-2 was provided by the National Cancer Institute Biological Resources Branch (Frederick, Md.). Phosphorothioate-backboned oligodeoxynucleotides (ODN; 25 bp) were synthesized with “machine mixed bases” by Integrated DNA Technologies. Anti-human CD28 antibody (clone CD28.2) was purchased from eBioscience and anti-human CD3e antibody (clone 64.1) was purified in-house. hTGFβ1 (10 ng/ml) was purchased from Peprotech. Complete media for cell culture was RPMI 1640 medium with 10% FBS, 100 units/mL penicillin, 100 mg/mL streptomycin, 1 mM nonessential amino acids, 50 mM beta-mercaptoethanol, 1 mM sodium pyruvate, and HEPES.

### Isolation, Stimulation, and Expansion of CD4 T Cells

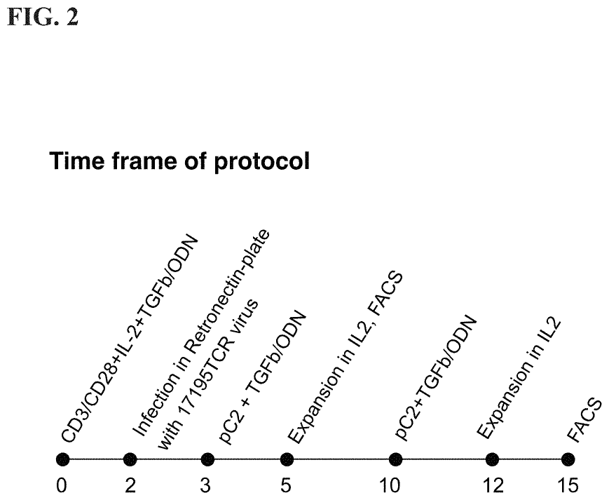

Buffy-coat fractions from healthy donors were provided by Department of Transfusion Medicine at the National Institutes of Health. All procedures were approved by Uniformed Services University of the Health Sciences Institutional Review Board, and all blood donors provided written informed consent in accordance with Declaration of Helsinki. Peripheral blood mononuclear cells (PBMCs) were isolated from buffy coats by ficoll separation (Ficoll-Paque Plus; GE Healthcare). T cells were enriched from PBMCs by CD4⁺ selection using human CD4 MicroBeads (Miltenyi Biotec Inc., Auburn, Calif.) according to the manufacturer's instructions. Enriched cells were stained and then sorted as T naïve (CD4⁺CD25^{-/lo} CD127⁺CD45RA⁺) and natural Treg (nTreg, CD4⁺CD25^{hi}CD127^{lo/-}) cells on a fluorescence-activated cell sorting (FACS) Melody cell sorter (BD, Franklin Lakes, N.J.). For sorting, anti-human CD4-FITC, anti-human CD25-PECy7, anti-human CD127-PE, and anti-human CD45RA-APC antibodies were purchased from Biolegend. TGFβ1 (10 ng/ml), ODN (2 μM), or both were maintained in the cultured wells for five days after pre-stimulation with anti-CD3 and anti-CD28 antibodies, and another five days after 2^{nd} activation with pC2 and PBMCs (FIG. 2, from day 0 to day 5, and day 10 to day 15). For transduction of 17195, sorted T naïve cells or nTregs were pre-stimulated with anti-CD3e (5 μg/mL) and anti-CD28 antibodies (2 μg/mL) for 48 hours in the presence of recombinant human IL-2 (200 IU/mL). Pre-stimulated cells were transferred into 17195-TCR retroviral particle-coated, retronectin (10 μg/mL)-pretreated plates and incubated for 24 hours. Then, transduced cells were restimulated with gamma-irradiated (6000 rad) HLA-DRB1*01:01 (“DR1”) PBMCs plus cognate FVIII C2 peptide 2191-2220 (1 μg/ml) plus IL-2 (200 IU/ml) until day 5 in the conditions as indicated. Ratio of T cells:irradiated PBMCs was 1:10. After day 5, cells were resuspended in fresh media and IL-2 and the cells were continuously cultured up to day 10. On day 10, the cells were activated again with antigen specific TCR stimuli and IL-2 until day 15.

### Production of Retroviral Particles and T Cell Transduction

Retrovirus containing the TCR-17195, which is the FVIII-2191-2220-specific TCR, was produced using a Phoenix-Ampho packaging system (Clonetech) and used for preparation of transduced T cells as previously described (Kim et al., Blood 125:1107-1115 (2015)).

### Intracellular Staining for Foxp3, Helios, and Cytokines

T cells differentiated in different conditions were stained with anti-CD4-PECy7 antibody (Biolegend) and fixed with 4% paraformaldehyde solution. Fixed cells were permeabilized in bovine serum albumin-containing 0.1% Triton X-100/phosphate-buffered saline (PBS). Permeabilized cells were stained with antibodies for Foxp3-APC and Helios-PE (Biolegend). For cytokine analysis, expanding cells (day 12) were rested in medium without IL-2 for three days and re-stimulated with PMA (12-O-Tetradecanoyl-phorbol-13-acetate, 50 ng/mL) and Ionomycin (1 μg/mL) for 4 hours in the presence of Golgistop (0.75 μl/mL) at 37° C. The cells were fixed, permeabilized, and stained for IL-2-PE (Biolegend) and IFN-rPECy7 (Biolegend). Stained cells were acquired on an LSRII instrument (BD) and analyzed using FlowJo software (Tree Star, Inc.).

### In Vitro Suppression Assay

17195TCR-transduced effector T cells were expanded for 3 weeks and then rested in IL-2-free media for 3 days. The effector cells (4×10⁴) were mixed with 17195TCR-transduced iTregs, which were expanded in different conditions of TGFβ1 and/or ODN, at indicated ratios, and followed by co-culture for 4 days in the presence of γ-irradiated DR1-PBMCs and FVIII C2 peptide-2191-2220 (0.5 μg/mL) without the addition of IL-2. For [³H]-thymidine incorporation assay, [³H]-thymidine was added to the cells 18 hours prior to harvest, and radioactivity (count-per-minutes, CPM) was measured using a scintillation beta counter.

### Example 2. Results

### Isolation of Naïve T Cells.

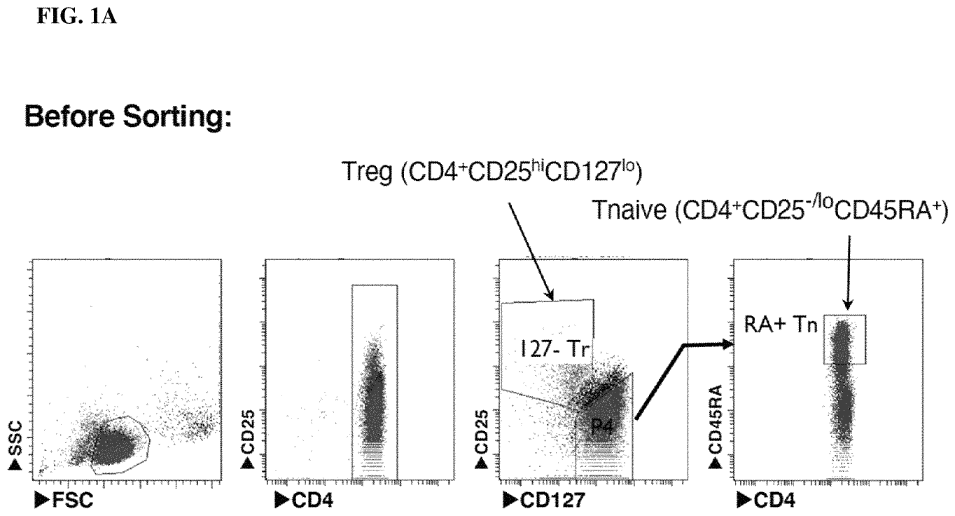

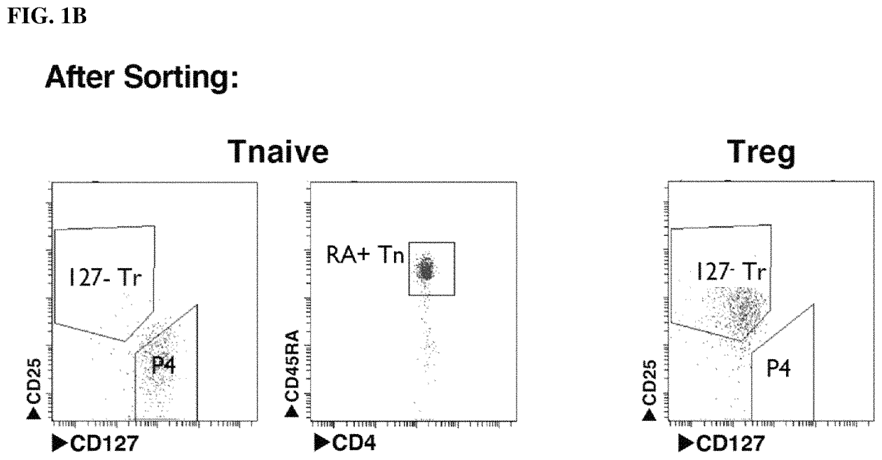

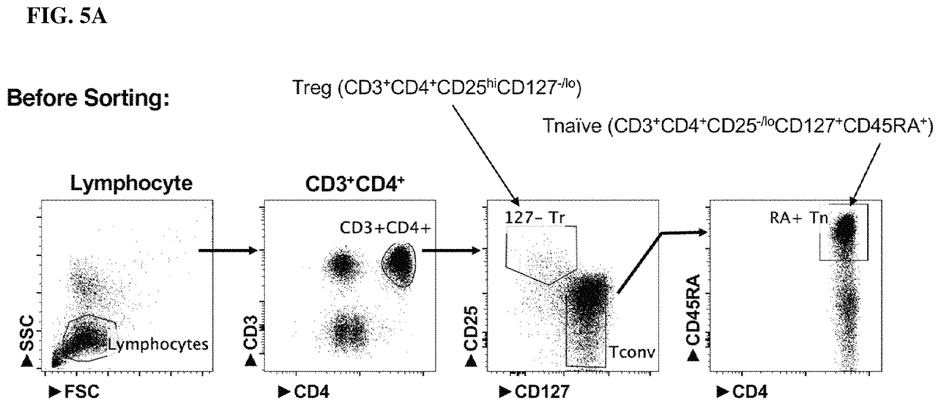

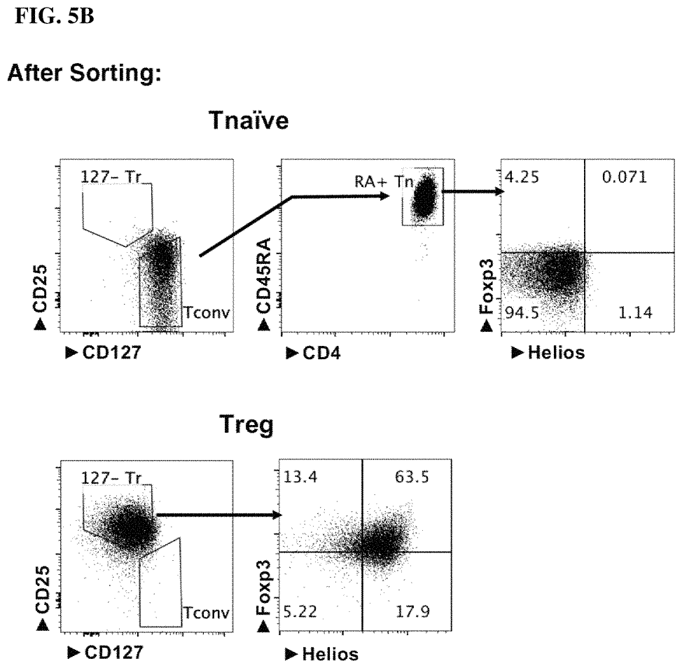

To compare the efficacy of different conditions of the iTreg induction, isolated CD4⁺ T cells were isolated from human peripheral blood using human CD4 microbeads. Positively selected CD4⁺ T cells were further analyzed and sorted into naïve T and nTreg subpopulations as shown in FIGS. 1A-1B and 5A-5B. “Lymphocyte Gate” was performed with expected morphology using forward versus and side scatter, followed by gating of the CD3⁺CD4⁺ T cell population to remove concomitant CD3⁻ or CD4⁻ cells from CD4⁺ bead isolation process. Next, CD4⁺ T lymphocytes were gated for the naïve T cells (CD3⁺CD4⁺CD25^{-/lo}CD127⁺CD45RA⁺) and regulatory T (nTreg, CD3⁺CD4⁺CD25^{hi}CD127^{-/lo}) cells. Each sorted fraction was further confirmed for their purities using the same sorting strategy.

Phenotypic Analysis of iTregs

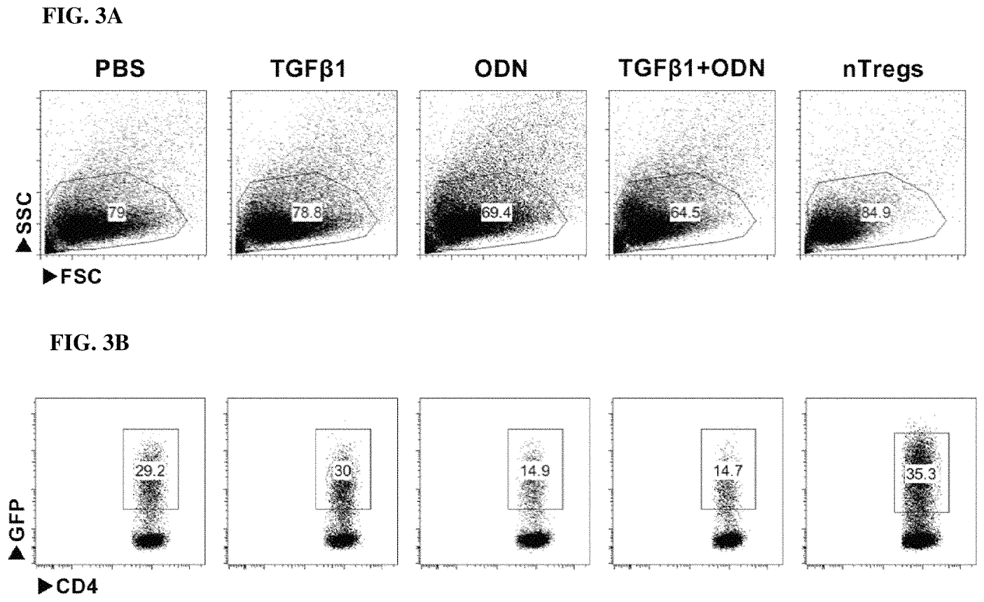

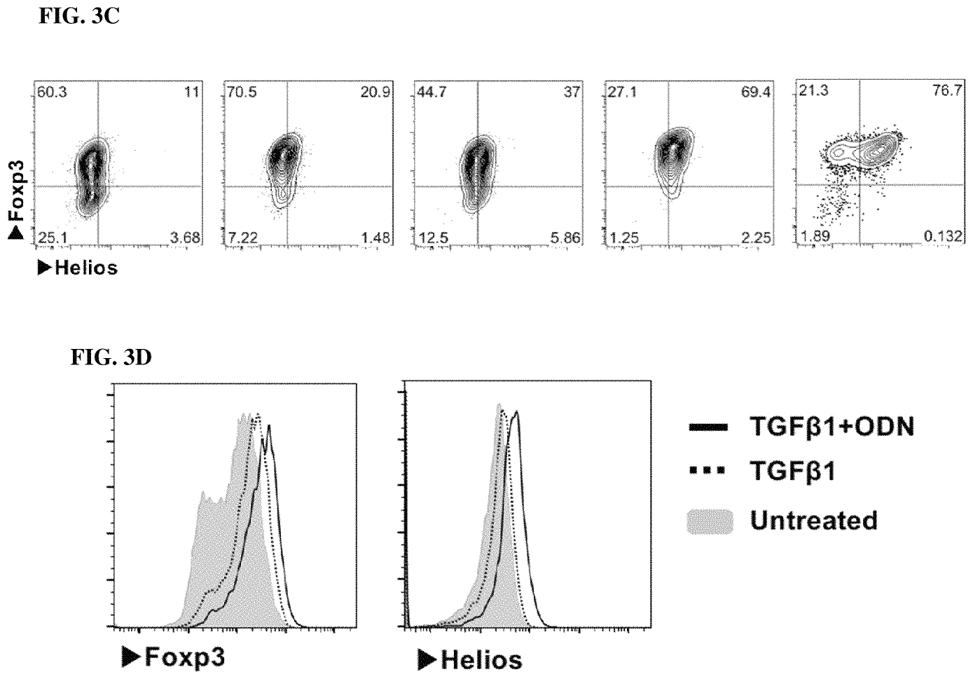

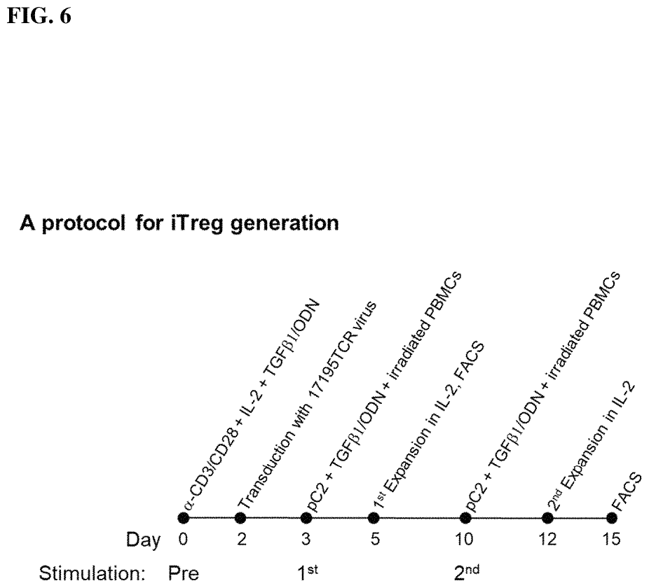

A novel protocol was developed to induce naïve T cells into Treg phenotype. FACS-sorted naïve T cells were pre-stimulated, transduced with retroviral 17195, and then stimulated with antigen-specific TCR stimuli and IL-2 (FIGS. 2 and 6). Lymphocyte gating and GFP^{+/-} gating strategy are shown in FIGS. 3A and 3B. Since 17195 TCR-transduced cells expressed GFP intracellularly (Kim et al., Blood 125:1107-1115 (2015)), antigenic stimulation will selectively activate GFP⁺17195 TCR transduced cells. For further Treg phenotypic analysis, GFP⁺ population was examined. Since Foxp3 alone is not sufficient to designate human Treg, an additional Treg-like signature molecule, Helios (another Treg associated-transcription factor that plays an important role in Treg function), was examined. On day 5, activated T cells under various treatments were analyzed for expression Foxp3 and Helios in GFP⁺17195 T cells.

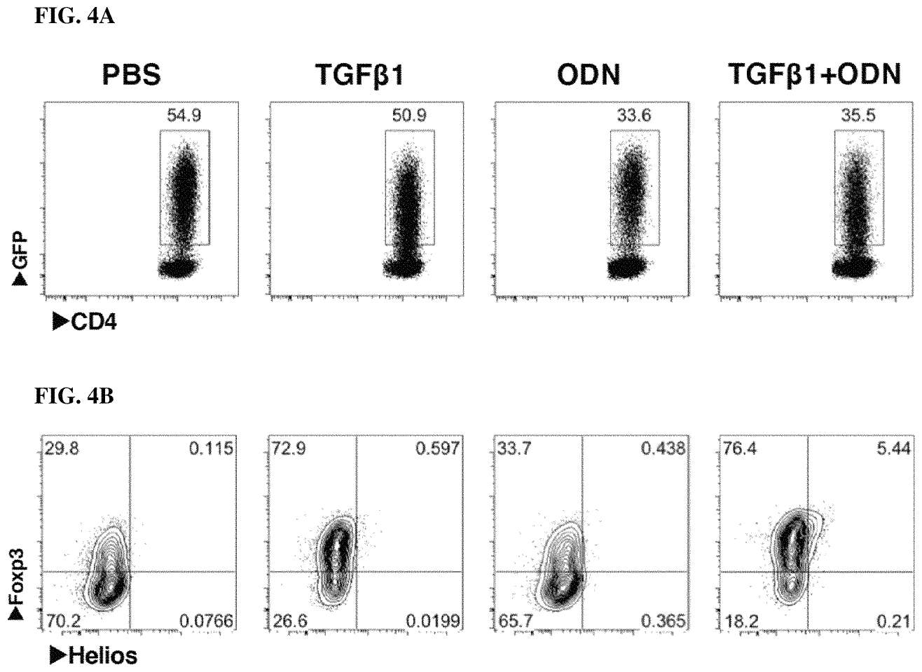

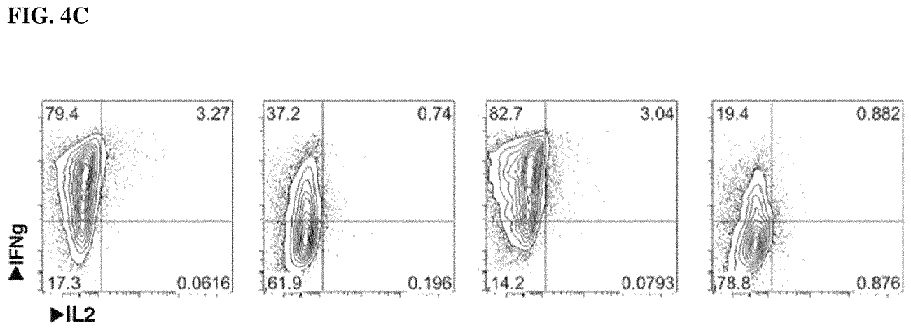

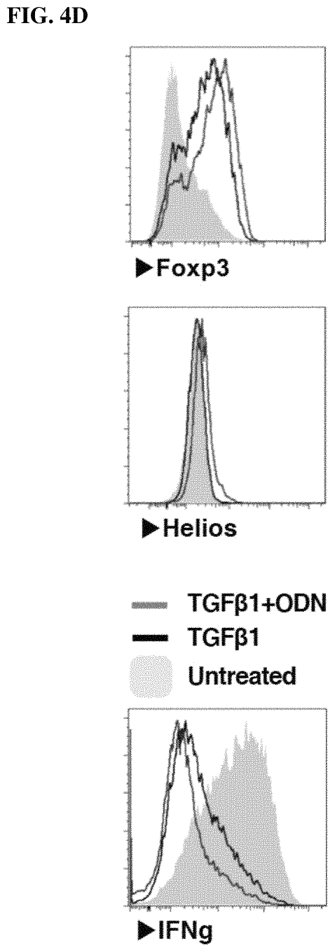

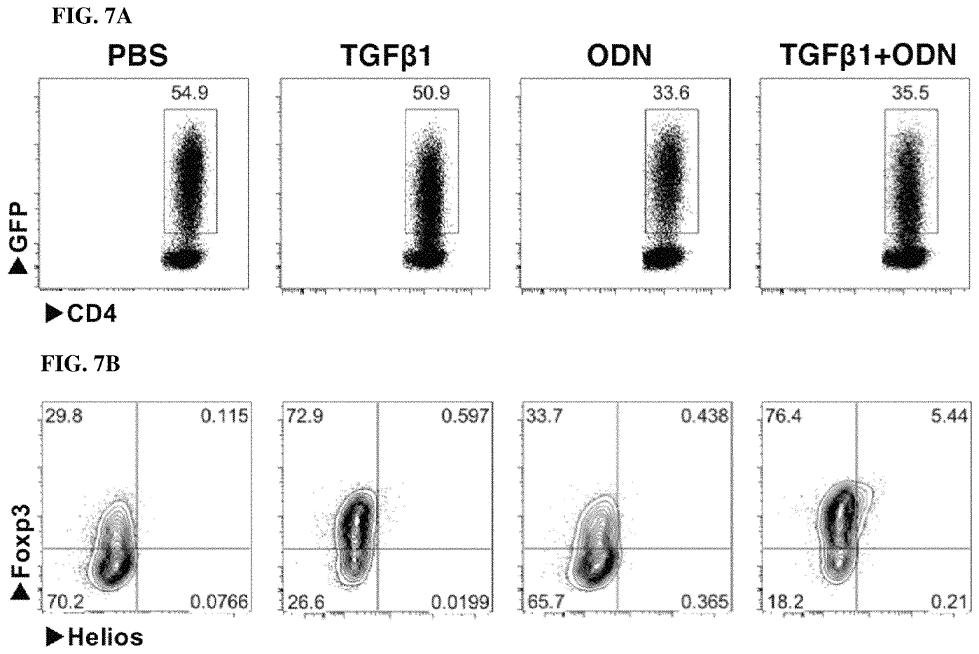

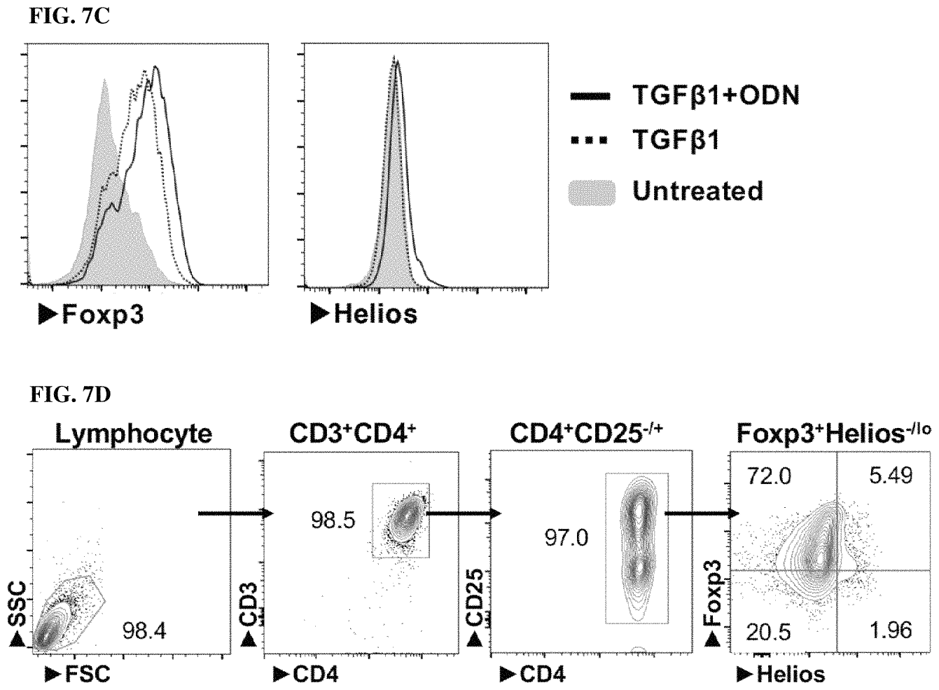

At day two (2) after antigenic peptide treatment, Foxp3 expression appeared in GFP-positive 17195 cells in all tested group as well as in nTreg group (FIGS. 3B and 3C). It is known that Foxp3 is transiently expressed in activated conventional T cells. Transient Foxp3 expression was decreased by further long-term expansion in the presence of PBS or ODN alone (FIGS. 4B-4D and 7B-7D).

At day two (2) after peptide treatment, GFP⁺ T cells treated with TGFβ1 alone induced Foxp3 expression marginally. The GFP⁺ cells treated with TGFβ1 and ODN together showed remarkable Foxp3 induction. Helios was slightly induced among the group as well (FIGS. 3C and 3D). However, Helios expression by treatment with TGFβ1 and ODN together was not strong enough to reach the expression level of Helios in nTreg cells (TGFβ1+ODN and nTreg in FIG. 3C). The result clearly indicated that TGFβ1 and ODN co-treatment leads T cells to acquire iTreg phenotypes, which is important to exert the immunosuppressive function.

Phenotypic analysis of Treg markers was also performed with 2^{nd} expanded cells. On day 10, 1^{st} expanded cells were re-stimulated with pC2 and γ-irradiated PBMCs in the indicated conditions in the presence of IL-2 for 15 days. Similar to what was seen with 1^{st} expanded cells (FIG. 3C), 2^{nd} expanded cells treated with both ODN and TGFβ1 retained the highest level of Foxp3 expression, compared to those treated with either ODN or TGFβ1 alone (FIGS. 4B, 4C, 7B, and 7C). However, the level of Helios expression weakly induced by the 1^{st} stimulation was disappeared during a long-term expansion of 15 days (FIGS. 4B and 7B). ODN alone treatment does not seem to increase Foxp3 expression compared to PBS treatment. The immunophenotype of iTregs produced by the methods disclosed herein was CD3⁺CD4⁺CD25^{-/+}Foxp3⁺Helios^{-/lo} (FIGS. 4D and 7D).

Cytokine Analysis of iTregs

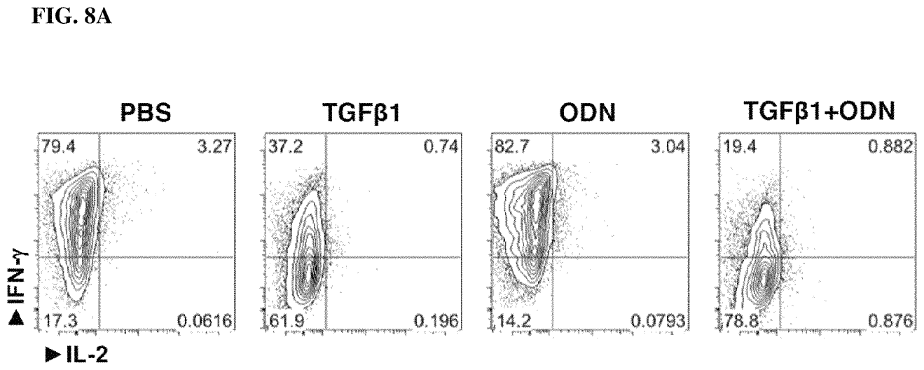

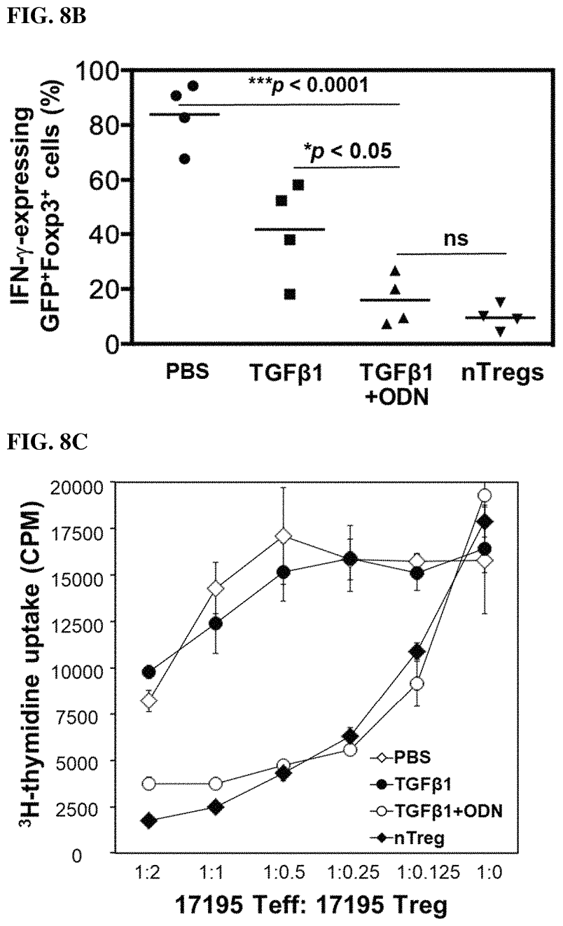

Contrary to mouse iTreg cells, Foxp3⁺ T cells by TGFβ1 treatment tends to express pathogenic inflammatory cytokines, such as IFN-γ or IL-2, which are critical indicators to represent immune responsive T cells, not suppressive Tregs. To address whether Foxp3⁺ cells by TGFβ1 and ODN acquire immunosuppressive function, the cytokine production was examined in the cells after a long-term expansion. On day 12, 17195TCR-transduced cells were rested for 3 days in culture without IL-2 and then re-stimulated with phorbol myristate acetate (PMA) and ionomycin. Cytokines-producing cells were measured by FACs analysis. IL-2-positive cells were low in all groups (FIG. 8A). However, stimulation with PMA and ionomycin induced a high fraction (˜80%) of IFNγ-expressing 17195 T cells in iTregs generated with PBS or ODN alone. iTregs generated by the TGFβ1 alone exhibited lower frequency (˜40%) of IFNγ-expressing cells, compared to iTreg by PBS or ODN alone. Significantly, TGFβ1 and ODN co-treatment ensured the lowest percentage (19.4%) of IFNγ-expressing 17195 T cells (FIG. 8A). The fraction of IFNγ-expressing 17195 T cells was as low as that of nTregs, when iTregs were generated with both TGFβ1 and ODN (FIG. 8B). Taken all together, cytokine production data clearly indicate that cotreatment of ODN and TGFβ1 grants Foxp3⁺ cells to suppress inflammatory cytokine productions.

Immunosuppression by iTregs

To test antigen-selective immunosuppression by iTregs, rested 17195TCR-transduced effector T cells were co-cultured with 17195TCR-transduced iTregs at various ratios with γ-irradiated DR1-PBMCs and FVIII C2 peptide in the absence of IL-2. 17195TCR-transduced iTregs, which were generated in the presence of TGFβ1 and ODN, successfully blocked the proliferation of 17195TCR T effectors as 17195TCR-transduced nTreg did (FIG. 8C). However, 17195TCR-transduced iTregs generated with PBS or TGFβ1 alone partially suppressed the proliferation of 17195TCR T effectors only at high Treg ratio (1:2 and 1:1) (FIG. 8C).

### CONCLUSION

A novel protocol of human iTreg generation using TGFβ1 and ODN from naïve T cells (CD3⁺CD4⁺CD25^{-/lo}CD127⁺CD45RA⁺) has been developed. TGFβ1 and ODN co-treatment induced Foxp3 expression, which is a signature nuclear factor of Treg cells. This co-treatment also induced to express Helios in initial activation stage, but the induction was reverted by a long-term expansion. Interestingly, Foxp3⁺ T cells by TGFβ1 and ODN together exhibited suppressed production of the inflammatory cytokines, IFNγ, implying that TGFβ1 and ODN co-treatment convert the T cells to suppressive iTreg cells. Furthermore, iTregs generated by TGFβ1 and ODN had a strong suppressive activity on the proliferation of T effectors, compared to iTregs by TGFβ1 alone. Importantly, a novel combination of TGFβ1 and ODN as a protocol has been identified to induce human iTregs with superior Treg phenotypes in vitro compared to the TGFβ1 alone. These results advance the understanding of the conditions for human iTreg production and have important implications for their therapeutic use for targeting autoimmune diseases. The effect of ODN is possibly additive to TGFβ1's iTreg function or prolonging a stability of Treg phenotypes.

The above description is for the purpose of teaching the person of ordinary skill in the art how to practice the subject of the present application, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

The breadth and scope of the present invention should not be limited by any of the above-described exemplary embodiments but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

1. A method of producing an immunosuppressive iTreg (induced regulatory T cell), comprising treating an isolated CD3⁺CD4⁺CD25^{-/lo}CD127⁺Foxp3⁻Helios⁻ T cell with an isolated oligodeoxynucleotide (ODN) having a phosphorothioate backbone and TGFβ1.

2. The method of claim 1, further comprising treating the T cell with IL-2.

3. The method of claim 1, wherein the ODN is 11-49 nucleotides in length.

4. The method of claim 3, wherein the ODN is 21-25 nucleotides in length.

5. The method of claim 4, wherein the ODN is 25 nucleotides in length.

6. The method of claim 1, wherein the iTreg is CD3⁺CD4⁺CD25^{-/+}Foxp3⁺Helios^{-/lo} and immunosuppressive.

7. The method of claim 6, wherein the iTreg expression of IFNγ is lower compared to treatment with TGFβ1 alone or ODN alone.

8. An iTreg produced by the method of claim 1.

9. An iTreg (induced regulatory T cell) that is CD3⁺CD4⁺CD25^{-/+}Foxp3⁺Helios^{-/lo} and immunosuppressive.

10. The iTreg of claim 9, wherein the iTreg expression of IFNγ is lower compared to treatment with TGFβ1 alone or ODN alone.

11. A composition comprising the iTreg of claim 9 and a carrier.

12. A composition comprising an isolated CD3⁺CD4⁺CD25^{-/lo} CD127⁺Foxp3⁻Helios⁻ T cell, an isolated oligodeoxynucleotide (ODN) having a phosphorothioate backbone, and TGFβ1.

13. The composition of claim 12, further comprising IL-2.

14. The composition of claim 12, wherein the ODN is 11-49 nucleotides in length.

15. The composition of claim 14, wherein the ODN is 21-25 nucleotides in length.

16. The composition of claim 15, wherein the ODN is 25 nucleotides in length.

17. A kit comprising an isolated CD3⁺CD4⁺CD25^{-/lo}CD127⁺Foxp3⁻Helios⁻ T cell and an isolated oligodeoxynucleotide (ODN) having a phosphorothioate backbone.

18. The kit of claim 17, further comprising TGFβ1.

19. The kit of claim 18, further comprising IL-2.

20. The kit of claim 17, wherein the ODN is 11-49 nucleotides in length.

21. The kit of claim 20, wherein the ODN is 21-25 nucleotides in length.

22. A method of treating an autoimmune disease in a subject in need thereof, comprising administering to the subject the iTreg of claim 9.

23. The method of claim 22, wherein the autoimmune disease is Type I diabetes, multiple sclerosis, Graft vs. host disease, allograft rejection, atopic dermatitis, psoriasis, inflammatory bowel disease, neuromyelitis optica, rheumatoid arthritis, alopecia areata, systemic lupus erythematosus, pemphigus vulgaris, autoimmune vasculitis, xenogenic organ transplantation, allogenic organ transplantation, or ADA (anti-drug antibody)-mediated complications.
